(19) United States
(12) Patent Application Publication
(10) Pub. No.: US 2023/0201122 A1
(43) Pub. Date: Jun. 29, 2023

(54) CHEMOEMBOLIC COMPOSITIONS AND METHODS OF TREATMENT USING THEM

(71) Applicant: Boston Scientific Medical Device Limited, Co. Gailway (IE)

(72) Inventors: Matthew Dreher, Rockville, MD (US); Bhanu Koppolu, Cary, NC (US); Steven Kangas, Woodbury, MN (US); Rhiannon Johnson, Maple Grove, MN (US); Yiqing Tang, Guildford (GB)

(73) Assignee: Boston Scientific Medical Device Limited, Co. Galway (IE)

(21) Appl. No.: 18/087,423

(22) Filed: Dec. 22, 2022

Related U.S. Application Data
(60) Provisional application No. 63/293,179, filed on Dec. 23, 2021.

(51) Int. Cl. A61K 9/16; A61K 31/502; C07K 16/28; A61K 45/06

(52) U.S. Cl. CPC A61K 9/1682; A61K 9/1647; A61K 31/502; C07K 16/2803; A61K 45/06

(57) ABSTRACT

The present disclosure relates to, inter alia, improved methods for the treatment of solid tumors using embolic polymer microspheres, to embolic polymer microspheres that comprise a polymer and an inhibitor of the enzyme poly ADP ribose polymerase (PARP inhibitor) wherein the PARP inhibitor is held within the polymer microsphere and is elutable from the microsphere in aqueous media, and to methods of loading embolic polymer microspheres.

### CROSS-REFERENCE TO RELATED APPLICATION

This application claims the benefit of priority of U.S. Provisional Application No. 63/293,179, filed Dec. 23, 2021, the entire disclosure of which is hereby incorporated by reference herein for all purposes.

### FIELD

The present disclosure relates to, inter alia, improved methods for the treatment of solid tumors, using embolic microspheres, to methods of loading embolic microspheres and to drug loaded embolic microspheres.

### BACKGROUND

Poly (ADP-ribose) polymerases (PARPs) are involved in DNA repair. They are activated by single and double-strand breaks and play a role in the base excision repair pathway. PARP inhibitors are presently approved (FDA) for ovarian, fallopian tube, peritoneal and breast cancers, but in other tumors their efficacy has been disappointing (see for example Zhang et al, Hepatology (2012) 55(6): 1840-1851 and Gabrielson et al, Cancer Chemother. Pharmacol. (2015) 76:1073-1079). PARP inhibitors have, for example, demonstrated efficacy against hepatocellular carcinoma (HCC) cell lines in vitro, although clinically, they have proven less effective.

Certain tumors, may be amenable to treatment by embolization procedures, in which an embolic material, such as a microsphere suspension, is introduced into a blood vessel supplying the tumor, where it lodges, causing an embolus that shuts down blood supply to local tissue. This approach is known as transarterial embolization or TAE. In one approach, a drug may be incorporated into the embolic material and is released into the tissues surrounding the embolus. This approach is known as transarterial chemoembolization or TACE. Embolic microspheres have been prepared from a variety of biocompatible materials, including both natural and synthetic polymers.

Embolization of tissue during TAE leads to ischemia and local tissue necrosis, which has been shown, in some cases to be associated with an increase in tumor antigen specific T-cells. For example, Lakshmana et al. (J. Immunol. (2017) 178(3): 1914-1922), noted that embolization was associated with an increase in peripheral alpha fetoprotein (AFP)-specific CD4⁺ T cells, which was associated with the induction of tumor necrosis and improved outcomes.

Tumor antigens recognized by T-cells are thought to derive from two main groups of proteins: firstly non-mutated proteins to which the T-cell tolerance is incomplete, or newly derived, and secondly, tumor specific, antigens arising from somatic mutations within the tumor genome; these are the so-called neoantigens. Neoantigens are believed to arise when these somatic mutations are improperly corrected by a compromised DNA damage repair process commonly found in tumor cells. Some tumor types appear to carry large numbers of such somatic mutations. In HCC for example the number of such mutations in individually analyzed tumors ranged from less than one to greater than 10 per megabase (see Schumacher and Schreiber, Science (2015) 348, 69-74.). Never-the-less, in some cancers at least, relatively few mutations appear to generate neoantigens for which a CD4⁺ or CD8⁺ T-cell reactivity can be detected.

### SUMMARY

The present inventors have conceived that locoregional delivery of PARP inhibitors to tumors in combination with an embolic event, such as that provoked by the delivery of a microsphere suspension, will potentially lead to an improved tumor response (such as but not limited to reduction in tumor size or improved overall survival) to the PARP inhibitor and thereby provides an improved method of treatment of the tumor or the patient.

The present inventors have further conceived that enhancement of DNA damage in concert with the presence of locally high levels of PARP inhibitors will potentially lead to a higher potential for the production of neoantigens.

The present inventors have further conceived that T-cell response to tumors (such as those of the liver including HCC) can potentially be improved by local delivery of PARP inhibitors such as olaparib, with such improved T-cell response leading to an improved treatment of the tumor. Further, this response can be augmented by additional treatments leading to DNA damage, such as radiation treatment and/or may be further exploited by combining the local delivery of PARP inhibitor (with or without further DNA damage promotors) with one or more immune checkpoint inhibitors.

In various aspects, the present disclosure pertains to polymer microspheres that comprise a polymer and a PARP inhibitor, wherein the PARP inhibitor is held within the polymer microsphere and is elutable from the microsphere in aqueous media.

In some embodiments, at least a portion of the PARP inhibitor is held within the microsphere by ionic interaction of the PARP inhibitor with the polymer and/or is physically entrapped within the polymer microsphere.

In some embodiments, which may be used in conjunction with the preceding aspects and embodiments, the PARP inhibitor is elutable from the polymer microsphere in an aqueous medium, such as phosphate buffered saline or water.

In some embodiments, which may be used in conjunction with the preceding aspects and embodiments, the PARP inhibitor may be selected from Olaparib (AZD-2281), Rucaparib (PF-01367338), Niraparib (MK-4827), Talazoparib (BMN-673), Veliparib (ABT-888), CEP 9722, E7016, BGB-290 and 3-aminobenzamide.

In some embodiments, which may be used in conjunction with the preceding aspects and embodiments, the polymer may have one or a combination of two or more of the following characteristics: (a) the polymer may be anionically charged at pH7.4, (b) the polymer may be crosslinked, (c) the polymer may be in the form of a hydrogel, or (d) the polymer may be biodegradable or bioerodible.

In some embodiments, which may be used in conjunction with the preceding aspects and embodiments, the polymer may be a polyester, a polysaccharide or a biodegradable, or bioerodible non-biodegradable/bioerodible PVA polymer. For example, the polymer may be a biodegradable or bioerodible crosslinked PVA polymer or copolymer, or the polymer may be a poly(lactide-co-glycolide) (PLGA) copolymer. In some of these embodiments, the ratio of lactide to glycolide units in the PLGA is between 50:50 and 10:90.

In some embodiments, which may be used in conjunction with the preceding aspects and embodiments, the polymer microsphere is a hydrogel polymer microsphere comprising a crosslinked polyvinyl alcohol polymer and a PARP inhibitor, where the crosslinked polyvinyl alcohol polymer has a negative charge at pH7.4, and the PARP inhibitor is held within the polymer microsphere and is elutable from the polymer microsphere in aqueous media. In some of these embodiments, the PARP inhibitor may be in a particulate form within the polymer microsphere.

In some embodiments, which may be used in conjunction with the preceding aspects and embodiments, the polymer microsphere comprises poly(lactide-co-glycolide) (PLGA) and a PARP inhibitor, where the PARP inhibitor is held within the polymer microsphere and is elutable from the microsphere in aqueous media (e.g., based at least in part upon biodegradation or bioerosion of the polymer microsphere).

In various additional aspects, the present disclosure pertains to medical compositions that comprise a plurality of polymer microspheres in accordance with any of the above aspects and embodiments.

In some embodiments, the medical composition is in the form of lyophilized powder that comprises the polymer microspheres. In some of these embodiments, the lyophilized powder further comprises a polyol protectant.

In some embodiments, the medical composition further comprises an aqueous solution within which the polymer microspheres are suspended as hydrated polymer microspheres.

In some embodiments, the medical composition comprises between 0.1 and 50 mg of PARP inhibitor/ml of hydrated polymer microspheres.

In some embodiments, which may be used in conjunction with the preceding aspects and embodiments, the medical composition is an injectable composition.

In various further aspects, the present disclosure pertains to methods for the treatment of a patient having a solid tumor, comprising delivering to the solid tumor a medical composition in accordance with the preceding aspects and embodiments, wherein the PARP inhibitor is eluted from the polymer microspheres and into the tumor tissue.

In some embodiments, the composition is delivered to the tumor by local injection.

In some embodiments, the composition is delivered to the tumor via one or more blood vessels feeding at least part of the tumor, and the polymer microspheres lodge in the blood vessels to provide an embolus. In some of these embodiments, the composition is delivered to the tumor via a microcatheter.

In some embodiments, which may be used in conjunction with the preceding aspects and embodiments, the treatment of the patient further comprises treatment with radiation therapy, which may be delivered before, during and/or after the delivery of the medical composition. For example, the radiation therapy may comprise external beam radiation therapy (EBRT), brachytherapy or selective internal radiation therapy (SIRT), among others.

In some embodiments, which may be used in conjunction with the preceding aspects and embodiments, the treatment of the patient additionally comprises delivering to the tumor one or more checkpoint inhibitors. The one or more checkpoint inhibitors may be delivered locally to the tumor or delivered systemically. The one or more checkpoint inhibitors may be delivered before, during and/or after the delivery of the medical composition.

In some embodiments, (a) the checkpoint inhibitor is selected from inhibitors of the binding of PD-1 to PD-L1, inhibitors of the binding of CTLA-4 to CD80 and/or CD86, or inhibitors of the binding of LAG-3 to MHC class II, or the inhibitors of the binding of TIGIT to CD112 and/or CD155 (b) the checkpoint inhibitor is selected from antibodies, or antigen binding fragments thereof, that bind to PD-1, PD-L1, LAG-3, TIGIT or CTLA-4, (c) the checkpoint inhibitor is selected from pembrolizumab, nivolumab, ipilimumab, atezolizumab, avelumab, tremelimumab, relatlimab, and durvalumab, (d) the checkpoint inhibitor is selected from antibodies, or antigen binding fragments thereof, that bind to TIM-3, or (e) the checkpoint inhibitor is selected from LY3321367, MBG453 and TSR-022.

In various additional aspects, the present disclosure pertains to methods for preparing an PARP inhibitor loaded hydrogel polymer microsphere comprising the steps of (a) contacting a solvent solution comprising PARP inhibitor dissolved in a first solvent with a dehydrated hydrogel polymer microsphere, (b) recovering the microsphere that is produced step (a), and (c) washing the recovered microsphere of step (b) with an second solvent, wherein the first solvent is an organic solvent suitable for dissolving PARP inhibitor at a concentration of at least 10 mg/ml at 25° C. and the second solvent is solvent in which PARP inhibitor is soluble at less than 0.1 mg/ml at 25° C. In some of these embodiments, the PARP inhibitor is olaparib.

In some embodiments, the first solvent is an organic solvent. Examples of organic solvents include, for example, dimethyl sulfoxide (DMSO), dimethyl formamide (DMF), N-methyl-2-pyrrolidone (NMP), dimethyl isosorbide (DMI), glycofurol, solketal, and combinations thereof, among others.

In some embodiments, the second solvent is an aqueous solvent, for example, water, normal saline, or phosphate buffered saline.

In various further aspects, the present disclosure pertains to PARP inhibitor for use in the treatment of a solid tumor, wherein the PARP inhibitor is provided in the form of a plurality of polymer microspheres comprising the PARP inhibitor and a polymer, including polymer microspheres in accordance with any of the preceding aspects and embodiments, and wherein the PARP inhibitor is held within the polymer microspheres and is elutable from the microsphere in aqueous media.

In various further aspects, the present disclosure pertains to the use of a PARP inhibitor in the manufacture of a medicament for the treatment of a solid tumor, wherein the PARP inhibitor is in the form of a plurality of polymer microspheres comprising the PARP inhibitor and a polymer, including polymer microspheres in accordance with any of the preceding aspects and embodiments, and wherein the PARP inhibitor is held within the polymer microsphere and is elutable from the microsphere in aqueous media.

### BRIEF DESCRIPTION OF THE DRAWINGS

FIG. 1 shows the size distribution of control non-loaded (bland) microspheres and loaded microspheres, in accordance with an aspect of the present disclosure.

FIG. 2 shows an elution profile of olaparib-loaded microspheres in a constant flow of warm saline (flow rate 1.7 mL/min at 37° C.) in terms of eluted olaparib concentration versus time, in accordance with an aspect of the present disclosure.

FIG. 3 shows an elution profile of olaparib-loaded microspheres in a constant flow of warm saline (flow rate 1.7 mL/min at 37° C.) in terms of total dose of olaparib eluted versus time, in accordance with an aspect of the present disclosure.

FIG. 4 shows an elution profile of olaparib-loaded microspheres in terms of total dose of olaparib eluted versus time under pseudo-sink conditions at 37° C., in accordance with an aspect of the present disclosure.

FIG. 5 shows an elution profile of olaparib-loaded PLGA microspheres in pH 7.4 PBS/tween 20 at 37° C. in terms of total drug release versus time, in accordance with an aspect of the present disclosure.

### DETAILED DESCRIPTION

In a first aspect, the present disclosure provides a polymer microsphere comprising an inhibitor of the enzyme poly ADP ribose polymerase (PARP) wherein the PARP inhibitor is held within the polymer microsphere and is elutable from the microsphere in aqueous media.

Beneficially, the PARP inhibitor is typically not covalently coupled to the polymer of the microsphere and is free to be eluted from the microsphere in aqueous media, particularly in ionic aqueous media such as blood plasma, normal saline, or phosphate buffered normal saline (0.01 M phosphate buffered saline (0.138 M NaCl; 0.0027 M KCl), pH 7.4).

The PARP inhibitor may be held within the microsphere by one or more non-covalent interactions (such as van der Waals forces, hydrophobic interactions and/or electrostatic interactions, including ionic interactions, also referred to as charge-charge or electrostatic interactions, charge-dipole interactions, and dipole-dipole interactions, including hydrogen bonding) with the polymer and/or physical entrapment within the microsphere. In various embodiments, the drug is distributed throughout a microsphere matrix, although it may be present in smaller quantities close to the surface due to washing processes during preparation. The drug may be for example, entrapped (e.g., as a powder and/or as a crystalline or amorphous form) within the polymer microsphere or may be held within the microsphere by non-covalent interaction(s) with the polymer, such as by an ionic interaction, particularly where the polymer and drug comprise a charged component.

The drug may be associated with the polymer microsphere in a number of ways, for example, by physical entrapment when the drug is mixed with the polymer during manufacture of the microsphere (for example, when the polymer is in the form of a monolithic biodegradable or bioerodible polyester matrix such as a poly(lactide co-glycolide) (PLGA) matrix. Alternatively the drug may be precipitated and/or crystallized within a polymer matrix of a pre-formed microsphere during a drug loading process (for example when the drug is loaded into a hydrogel polymer, such as crosslinked PVA (see for example WO2007/090897A1 and WO2007/085615A1).

Holding the drug within the polymer microsphere by ionic interaction is particularly useful where the drug carries, or can be induced to carry, a charge, for example by inducing the drug to form a salt by exposure to an acidic or basic solution as appropriate. Such loading mechanisms are useful where the polymer microsphere carries a charge at physiological pH (pH 7.4). Drug loading by precipitation and/or crystallization is useful where the drug has a low solubility in water.

In use, once the microsphere is delivered, the drug will be released from the microsphere into the surrounding medium (tissues or blood) for example by bioerosion or biodegradation of the polymer forming the microsphere, by diffusion of the drug from the polymer matrix of the microsphere, or a combination of both. For the avoidance of doubt, the term “elution” is considered, herein, to encompass each or both of these mechanisms of release. Release of the drug may be verified at physiological pH (7.4) and/or in an ionic medium. Typically in phosphate buffered normal saline.

The polymer(s) forming the microsphere may be a natural polymer, a synthetic polymer, or a hybrid of a natural polymer and a synthetic polymer. Any polymer which is suitable to hold a drug within the polymer microsphere in such a way that it is elutable from the microsphere in aqueous media, such as phosphate buffered normal saline, may be used.

Suitable natural polymers include proteins (such as gelatin), polysaccharides (such as starches, chitosans, glycogens, celluloses, such as methyl celluloses, carboxymethylcelluloses, or hydroxyethylcelluloses, alginates, and polysaccharide gums, such as carrageenans, guars, xanthans, gellans, locus bean gums and gum arabics).

Suitable synthetic polymers include acrylates, acrylamides, acrylics, acetals, allyls, synthetic polysaccharides, methacrylates, polyamides, polycarbonates, polyesters, polyethers, polyimides, polyolefins, polyphosphates, polyurethanes, silicones, styrenics, and vinyls, or combinations and/or copolymers thereof. In some embodiments, the polymer is a homopolymer or copolymer that comprises one or more monomers selected from: vinyl alcohols, ethylene glycol (ethylene oxides), propylene glycols (propylene oxides), acrylates, methacrylates, acrylamides or methacrylamides.

In some embodiments, the polymer may be a natural and/or synthetic hydrophilic polymer because of their improved biocompatibility. Suitable hydrophilic polymers include polyvinyl alcohols, acrylates and methacrylates, and their salts, such as polyacrylic acids, polymethacrylic acids, and polymethylmethacrylates, carboxymethylcelluloses, hydroxyethylcelluloses, polyvinylpyrrolidones, polyethylene glycols (PEG), PEG-methacrylates, PEG-methylmethacrylates, polyacrylamides such as N,N-methylene-bis-acrylamides or tris(hydroxymethyl)methacrylamides, or a natural polymer such as a protein or polysaccharide, such as those discussed herein or elsewhere or a combination or co-polymer comprising at least one of the foregoing.

In some embodiments the hydrophilic polymer comprises or is a polyhydroxylated polymer, i.e. a polymer that comprises repeating units bearing one or more pendant hydroxyls. Preferred polyhydroxylated polymers include those comprising polyol esters of acrylates and methacrylates, including poly(hydroxyalkylacrylates) and poly(hydroxyalkylmethacrylates), such as poly(hydroxyethylmethacrylate); poly(hydroxyalkylacrylamides) and poly(hydroxyalkyl methacrylamides), such as tris(hydroxymethyl)methacrylamide; poly(PEGacrylates) and poly(PEGmethacrylates); polymers comprising vinyl alcohols such as poly(vinyl alcohol) polymers or (ethylene-vinyl alcohol) copolymers; and polysaccharides.

In a further embodiment, the hydrophilic polymer may be a polycarboxylated polymer i.e. a polymer that comprises repeating units bearing one or more pendant carboxyl groups. These polymers include, for example, polyacrylic acids polymethacrylic acids and their copolymers and their salts, such as Group I or Group II metal salts, e.g., sodium, potassium, calcium or magnesium salts.

In one embodiment the hydrophilic polymer is in the form of a hydrogel, in which the polymer is crosslinked, either covalently or non-covalently. These polymers are water-swellable but water-insoluble. The polymer may comprise greater than 50% water by weight, for example up, to 99% water by weight. Such polymers may comprise, for example 60% to 98% water by weight.

In some embodiments, the polymer may be a biodegradable or bioerodible polymer. Biodegradable or bioerodible polymers include, but are not limited to, polyesters and polysaccharides (such as those mentioned elsewhere herein).

Biodegradable and/or bioerodible polyesters include, but are not limited to polylactides such as polylactide (PLA), polyglycolides such as or polyglycolide (PLG), polyhydroxyalkanoates such as polyhydroxybutyrate, or polyhydroxyvalerate, poly ε-caprolactone, and co-polymers of the foregoing such as poly(lactide-co-glycolide) (PLGA) or poly(3-hydroxybutyrate-co-3-hydroxyvalerate), among others. In one preferred embodiment the biodegradable polymer is PLGA.

Further examples of biodegradable or bioerodible polymers are polyvinyl alcohol (PVA) polymers crosslinked with crosslinkers comprising a disulphide linkage (e.g., WO2012/101455, incorporated herein by reference) and PVA polymers whose PVA backbone is crosslinked by C₃ to C₈ diacid, such as α-ketoglutarate, through ester linkages with hydroxyl groups of the PVA backbone (e.g., WO2020/003152, incorporated herein by reference).

In some embodiments, the polymer may comprise groups that are charged at pH 7.4. Such groups may carry positive or negative net charges, which are able to reversibly bind compounds carrying the opposite net charge at physiological pH (pH 7.4). A variety of charged groups may be used, including sulphonate, phosphate, ammonium, phosphonium and carboxylate groups; carboxylate and sulphonate groups are preferred in some embodiments. Polymers which are anionically charged at pH 7.4. are preferred. Polymers that are charged at pH 7.4 may be selected from a range of polymers, which may be, for example, natural and/or synthetic, hydrophilic and/or hydrophobic, biostable and/or biodegradable, including those described above.

In some embodiments, the polymer of the polymer microspheres may be crosslinked. Crosslinking may be based on covalent crosslinking, noncovalent crosslinking, or a combination of both. Noncovalent crosslinking includes physical crosslinking for example by entanglement of polymer chains, or by the presence of crystal regions. Noncovalent crosslinking further includes ionic crosslinking. Ionic crosslinking can occur where charged groups on the polymer are crosslinked by groups carrying the opposite charge. In some cases this can be through divalent metal ions or metal ions of higher valency (trivalent, etc.), such as calcium, magnesium or barium, among others.

Covalent crosslinking can be achieved by any suitable method for covalently linking functional groups on different chains together. If crosslinking is achieved during the polymerization stage this can be by incorporation of a bifunctional monomer during polymerization. Alternatively or in addition, crosslinking may be achieved post-polymerization, for example, by a reacting a bifunctional species capable of reacting with functional groups on a preformed polymer, such as the hydroxyl or carboxyl groups, among others.

The crosslinkers may also introduce degradable regions (see, for example, WO2001/68720), either within the crosslinker or at the termini of the crosslinker, such as ester bonds (e.g., WO2020/003152)

In one embodiment the polymer comprises polyvinyl alcohol (PVA), such as homopolymers and co-polymers of PVA and particularly where such polymers are crosslinked and further where they are charged at pH 7.4.

One type of PVA polymer is a polyvinyl alcohol macromer, having more than one ethylenically unsaturated pendant group per molecule, which is formed by reaction of a PVA with ethylenically unsaturated monomers. The PVA macromer may be formed, for instance, by providing a PVA polymer with pendant ethylenically unsaturated groups, for example, pendant vinylic or acrylic groups. Pendant acrylic groups may be provided, for instance, by reacting acrylic or methacrylic acid with PVA to form ester linkages through a portion of the hydroxyl groups. Ethylenically-unsaturated-group-bearing compounds capable of being coupled to polyvinyl alcohol are described in, for instance, U.S. Pat. Nos. 4,978,713, 5,508,317 and 5,583,163. In a preferred embodiment, the macromer comprises a backbone of polyvinyl alcohol to which is coupled an (alk)acrylaminoalkyl moiety. One example of such a polymer comprises a PVA-N-acryloylaminoacetaldehyde dimethylacetal (NAAADA) macromer, known as Nelfilcon-B or acrylamide-PVA.

In one preferred embodiment this macromer may be reacted with ethylenically unsaturated monomers optionally bearing a positive or negative charge, such as 2-acrylamido-2-methylpropane sulfonic acid (AMPS) in order to crosslink the polymer.

Such polymers and methods of making them are described in WO0168720 and WO04071495. Microspheres of such polymers are commercially available as DC Bead®, LC Bead®, Bead Block® or their radiopaque version DC Bead® LUMI or LC Bead® LUMI (Biocompatibles UK Ltd).

The microspheres can be prepared in any desired size range, however, sizes ranging from about 10 μm (microns) to 2000 μm are typically preferred. Smaller sizes may pass through the microvasculature and lodge elsewhere beyond the embolization site. In most applications it will be desirable to have a small size range of microspheres in order to reduce clumping during delivery and provide predictable embolization. Microspheres may be sized before or after loading. For example, microspheres may be sized after loading to provide more accurate size ranges, should the loading process alter the sizing of the unloaded (also referred to as native or bland) microspheres. Typically, the microspheres have a mean diameter size range of from 10 to 2000 μm, more typically 20 to 1500 μm and even more typically, 40 to 900 μm. Preparations of microspheres typically provide microspheres in size ranges to suit the planned treatment, for example 100-300, 300-500, 500-700 or 700-900 microns. Smaller microspheres tend to pass deeper into the vascular bed and so for certain procedures, microspheres in the range 40-75, 40-90 or 70-150 microns are particularly useful.

The hydrogel microspheres may be provided in a dried form. Where microspheres are provided in dried form, it is advantageous to incorporate a pharmaceutically acceptable water-soluble polyol into the polymer before drying. This is particularly advantageous for hydrogels as it protects the hydrogel matrix in the absence of water. Useful polyols are freely water-soluble sugars (such as mono- or di-saccharides) and sugar alcohols, including glucose, sucrose, trehalose, mannitol and sorbitol.

The microspheres may be dried by any suitable process, however, drying under vacuum, such as by freeze drying (lyophilization) is advantageous as it allows the microspheres to be stored dry and under reduced pressure. Preferably such microspheres have a water content of less than 1% wt/wt and more preferably less than 0.1% wt/wt. Storing under vacuum leads to improved rehydration as discussed in WO2007/147902 (which is incorporated herein by reference). Typically, the pressure under which the dried microspheres are stored is less than 1 mBar (gauge).

Microspheres may be imageable to assist in visualization during or post procedure. Imageability is desirable for techniques that include but are not limited to the following: ultrasound, X-Ray, magnetic resonance imaging, superparamagnetic resonance imaging, positron emission imaging (such as PET), or SPECT (Single Photon Emission Computed Tomography). Imageability can enhanced in various embodiments by including a suitable contrast agent in the microsphere or any media surround the microsphere.

In a preferred embodiment the microsphere is imageable by X-ray. This can be enhanced by incorporating a radiopaque component as a contrast agent into the microsphere either covalently or non-covalently. Examples of non-covalently incorporated radiopacifying components include, for example particulate materials, such as barium salts such as sulphate (see, for example Thanoo et al J. Appl. Biomater. (1991) 2: 67-72) or metals such as tantalum, or iodinated oils such as Lipiodol® (e.g. EP1810698A1). The microspheres may comprise covalently coupled radiopacifying component, such as iodine or bromine (e.g., WO2015/033093) or bismuth (e.g., WO2018/093566). In one embodiment the microspheres comprise a radiopacifying agent in the form of covalently coupled iodine atoms. In one embodiment such microspheres comprise PVA as described elsewhere herein, and the radiopacifying agent is covalently bound to the PVA backbone. Commercially available radiopaque microspheres having covalently coupled iodine atoms include DC Bead Lumi® (Biocompatibles UK Ltd, Camberley, UK).

Poly ADP ribose polymerase (PARP) is a family of proteins involved in a number of cellular processes including DNA repair, genomic stability, and programmed cell death. The family includes PARP-1 and PARP-2 and several others. Inhibitors of the enzyme are well-known, and several have received FDA approval and are available commercially. Compounds for use in the microspheres of the present disclosure preferably inhibit one or more enzymes of the PARP family and preferably inhibit either one or both of PARP-1 and PARP-2. Known PARP inhibitors include, for example, olaparib (AZD-2281), rucaparib (PF-01367338) commercially available as the camsylate, niraparib (MK-4827), talazoparib (BMN-673), veliparib (ABT-888), CEP 9722, E7016 (GPI-21016), BGB-290, 2X-121, ABT-767, AZ-0108, JPI-547 (NOV 1402), NMS-P118, NMS-P293, NT-125. Others are believed to be in the pipeline. Olaparib is preferred in some embodiments.

PARP inhibitors may be loaded into the microspheres in a number of ways. The drug can be entrapped in the polymer by mixing with the polymer during manufacturing. Alternatively, the drug may be precipitated and/or crystallized within the polymer matrix. This approach is useful where the PARP inhibitor is of low water solubility, for example <0.1 mg/mL in water at 25° C. Loading may be achieved by contacting the microsphere with an organic solvent suitable for dissolving the PARP inhibitor, so as to imbibe the microsphere with the solvent. This can be achieved by either several washes of a microsphere in the solvent, to exchange any aqueous suspension medium, or by swelling a lyophilized microsphere in the solvent. The solvent-swollen microsphere is then contacted with a solvent solution of the PARP inhibitor, typically in the same solvent. This solvent for the PARP inhibitor is typically capable of dissolving the PARP inhibitor in an amount of at least 10 mg/ml at 25° C., preferably at least 20, 30 or 40 mg/ml at 25° C. Embodiments where the drug is dissolved at greater than 70 mg/ml at 25° C. are favorable. Microspheres are then recovered after a suitable period of equilibration, (e.g., 15 mins to 1 hr) and briefly washed (e.g., 0.5 to 1 minute) in a medium where the drug is soluble at less than 0.1 mg/ml. Aqueous solvents are typically used for this purpose and may be, for example, normal saline or water. The microspheres may then be freeze dried. This loading process is particularly useful for hydrogel microspheres and may be used whether or not the microsphere is ionically charged. The above loading process is suitable, for example, for olaparib.

Where the PARP inhibitor has net charge, the drug can be loaded into microspheres having an opposing net charge by contacting an aqueous solution of the drug with a suspension of microspheres until the microspheres have absorbed the drug, typically this process takes between 5 minutes and an hour. The microsphere is then recovered and may be lyophilized if required.

The quantity of drug loaded into the microspheres can be controlled, for example, by altering the amount or concentration of drug in the loading solution and/or by altering the number of available charged groups on the microsphere, depending on the loading method used and solubility and/or charge on the specific PARP inhibitor used. The level of drug in the microspheres is preferred to be in the range of 0.1 to 100 mg/ml of microspheres (fully swollen in normal saline), preferably 1 to 50 mg/ml.

The PARP inhibitor may be provided in the form of dried microspheres as described above, which may be formulated into an aqueous composition prior to use (using water for injection or normal saline for example), or may be in the form of microspheres in an aqueous composition, such as water for injection or normal saline. Prior to injection a contrast agent may be added to enhance visualization during administration. Such compositions provide a further aspect of the present disclosure.

For the purposes of visualizing the composition during delivery and/or adjusting the density of the composition, the composition may additionally comprise a contrast agent, suitable for use in an imaging modality including but not limited to X-ray, ultrasound, PET, SPECT, magnetic resonance imaging, superparamagnetic resonance imaging, nuclear medicine techniques. This may be, for example an iodine containing contrast agent, which may be ionic or non-ionic, but is preferably non-ionic.

In a further aspect, the disclosure provides a method for the treatment of a patient having a solid tumor, comprising delivering to the solid tumor a composition comprising a plurality polymer microspheres, for example, hydrophilic polymer microspheres, comprising a PARP inhibitor, wherein the PARP inhibitor is held within the polymer microspheres and is elutable from the microspheres in aqueous media, and wherein the PARP inhibitor is eluted from the microspheres into the tumor tissue. The dose may range from 1 mg/day or less to 50 mg/day or more, in some embodiments, ranging from 2.5 mg/day to 25 mg/day, for example, ranging from 5 mg/day to 10 mg/day in some cases.

The treatment provided by the disclosure has broad applicability in solid tumors. The composition comprising the microspheres may be delivered by the transarterial route in TACE, where the composition is delivered to the tumor via one or more blood vessels feeding at least part of the tumor and wherein the microspheres lodge in the blood vessels to provide an embolus. Alternatively, or additionally, microspheres may be delivered by direct injection into the tumor, for example, for the formation of a depot of drug within the tumor.

The method has particular use in tumors where a suitable microcatheters can be placed to deliver the microspheres selectively into blood vessels supplying the tumor, such as so called “hypervascular tumors”. Useful catheters for this purpose typically range from 2.4 to 2.8 Fr in outer diameter (OD). Specific tumors include liver tumors such hepatocellular carcinoma (HCC) and metastases deriving from neuroendocrine tumors (NETs) and colorectal cancer (mCRC) in the liver and prostate tumors. Other tumors include renal tumors, adrenal tumors, tumors of the brain, such as gliomas lung tumors, pancreatic tumors, head and neck tumors, ovarian tumors, breast tumors and testicular tumors.

Depending on the size and/or vascularity of the tumor, at least about 0.1 ml of the composition comprising the microspheres are delivered to the tumor, preferably at least about 0.5 mls. The maximum amount is typically dependent, in the case of TACE treatments, on the size of the tumor, its vascularity and the volume of vessels available for embolization. Typically the volume of the microsphere composition delivered is between about 0.1 and about 5 mls, more typically between about 0.2 and about 3 mls. Once the first vascular bed is embolized, the microsphere composition may be delivered to a second vessel supplying the tumor and so on. The figures given relate to the total volume of microsphere composition delivered to the tumor.

The treatment may further comprise treatment with radiation therapy, applied as External Beam Radiation Therapy (EBRT) or as Selective Internal Radiation Therapy (SIRT) or brachytherapy. The level of radiation used will depend on the sensitivity to damage, but will generally be in the range 10-120 Gy. The radiation therapy may be delivered before, during and/or after the delivery of the microspheres.

The method may further comprise treating the patient with one or more immune check point inhibitors. These inhibitors may be delivered before, during and/or after the delivery of the microspheres. Further, they may be delivered locally (i.e., intratumorally) or systemically. In one embodiment the polymer further comprises a checkpoint inhibitor that is held within the polymer microsphere and is also elutable from the microsphere in aqueous media. Treatment with one or more checkpoint inhibitors may take place in combination with radiation therapy.

Checkpoint inhibitors are well-known, and several are presently approved for use; others are in development. The checkpoint inhibitor may be selected from one or more of the following: inhibitors of the binding of PD-1 to PD-L1, inhibitors of the binding of CTLA-4 to CD80 and/or to CD86, inhibitors of the binding of LAG-3 to MHC-class 2 molecules and inhibitors of the binding of TIGIT to CD-112. Typically the inhibitor will be selected from antibodies (particularly humanized or fully human monoclonal antibodies), or antigen-binding fragments thereof, that bind to PD-1, PD-L1, LAG-3, TIGIT or CTLA-4. These include. inter alia, pembrolizumab, nivolumab, ipilimumab, atezolizumab, avelumab, tremelimumab, relatlimab and durvalumab, etigilimab, domvanalimab, tiragolumab, vibostolimab, EOS-448, ASP837414, BMS-986207. The checkpoint inhibitor may also be selected from antibodies, or antigen binding fragments thereof, that bind to TIM-3, such as LY3321367, MBG453 and TSR-022.

The checkpoint inhibitor is typically delivered according to the manufacturer's recommendations but for guidance, these will generally be in the range 0.5 to 25 mg/kg. For example:
Pembrolizumab 1 to 3 mg/kg preferably 2±0.5 mg/kg
Nivolumab 0.5 to 5 mg/kg preferably 1 to 4 mg/kg
Ipilimumab 0.1 to 20 mg/kg IV, preferably 1 to 10 mg/kg
Atezolizumab 0.5 to 2 mg/kg, preferably 1.5±0.5 mg/kg
Avelumab 10±5 mg/kg
Tremelimumab 0.5 to 5 mg/kg, preferably 1±0.5 mg/kg
Durvalimumab 5 to 30 mg/kg preferably 20±5 mg/kg

In a further aspect of the present disclosure, a PARP inhibitor is provided for use in the treatment of a solid tumor, wherein the PARP inhibitor is held within a plurality of hydrophilic polymer microspheres and is elutable from the microspheres in aqueous media.

In a further aspect of the present disclosure, the use of a PARP inhibitor in the manufacture of a medicament for the treatment of a solid tumor is provided, wherein the PARP inhibitor is held within a plurality of hydrophilic polymer microspheres and is elutable from the microspheres in aqueous media.

### Example 1: Loading Olaparib into PVA Hydrogel Microspheres

The following examples use samples of DC Bead LUMI® of various size ranges (Biocompatibles UK Ltd). This hydrogel microsphere comprises crosslinked PVA carrying a negative charge. The microspheres are radiopaque by virtue of an iodinated phenyl group covalently coupled to the PVA backbone.

1 mL of hydrated microspheres (DC Bead LUMI®, size range 70-150 μm), were washed with DMSO (5 mL×3) to replace the hydration medium. The volume of microspheres increased with DMSO washing, but stabilized after the third wash. Two milliliters of a solution of olaparib in DMSO (120 mg/mL) was mixed with the microspheres, and allowed to equilibrate for 1 hr. The olaparib solution was then removed and the microspheres washed with saline (15 mL×5), and sieved with a 40 μm cell strainer to remove drug particulates. Finally the washed microspheres were freeze-dried overnight, and free flowing microspheres were obtained. Table 1 shows the loaded dose calculated following DMSO extraction and analysis by HPLC against an olaparib standard.

TABLE 1

| | Loading dose (mg/mL | Loading yield |
|---|---|---|
| Sample | microspheres) | (%) |
| 1 | 17.0 | 10.6 |
| 2 | 21.5 | 13.4 |
| 3 | 22.3 | 13.9 |

### Example 2: Loading Olaparib into Freeze-Dried PVA Hydrogel Microspheres

Five milliliters of hydrated microspheres (DC Microsphere LUMI® size range 70-150 μm, Biocompatible UK Ltd) were lyophilized to remove water. The microspheres were mixed with 5 mL of a solution of olaparib in DMSO (80 mg/mL) and roller-mixed for 1 hr. After removing the drug solution, the microspheres were washed with saline (50 mL×3), followed by dispensing into 4 vials (1.5 mL per vial). The microspheres were then lyophilized for 24 hours and free flowing microspheres were produced. The loading yields were determined as per Example 1. The residual drug in solution was measured and the loading yield was calculated as the ratio between drug loaded in the microspheres and initial total addition of drug. The results are given in Table 2.

TABLE 2

| | Loading dose (mg/mL | Loading yield |
|---|---|---|
| Sample | microspheres) | (%) |
| 1 | 17.7 | 26.6 |
| 2 | 17.8 | 26.7 |
| 3 | 17.5 | 26.3 |
| 4 | 15.7 | 23.6 |
| 5* | 45.0* | 37.5* |

*Sample 5 was loaded by using 1 mL of microspheres with 1 mL of 120 mg/mL olaparib solution.

### Example 3. Bulk Loading of Olaparib into PVA Hydrogel Microspheres

40 mL of wet DC Bead LUMI® (40-75 uM) were transferred to a Duran bottle with excess packing solution removed. The microspheres were then washed twice in 150 mL of DMSO by agitation on a plate shaker at 400 rpm for 5 minutes. Forty milliliters of 80 mg/mL olaparib in DMSO were then added to the microsphere slurry, which targeted a maximum theoretical loading dose of 80 mg/mL microspheres. The loading solution/microsphere mixture was agitated at 400 rpm on a plate shaker for 1 hour. Once agitation was complete, the excess loading solution was removed. The loaded microspheres were then washed in 300 mL of 0.9% saline and agitated for 5 minutes at 400 rpm, followed by saline removal. This step was repeated for second saline washing. The loaded microspheres were dispensed into vials (1.5 mL per vial) and freeze dried. The lyophilized microspheres were then gamma sterilized at a dose of 25 kGy.

Loading efficiency of the microspheres was assessed using a DMSO/saline extraction and analysis by HPLC. The results are given in Table 3. Average volume of microspheres in vial after lyophilization determined to be 0.79 ml.

TABLE 3

| | | Loading dose |
|---|---|---|
| | Loading dose | mg/ml |
| Sample | (mg/vial) | microspheres |
| 1 | 26.66 | 33.75 |
| 2 | 25.98 | 32.88 |
| 3 | 26.51 | 33.56 |
| 4* | 26.83 | 33.96 |

*Sample 4 was extracted in 100% DMSO.

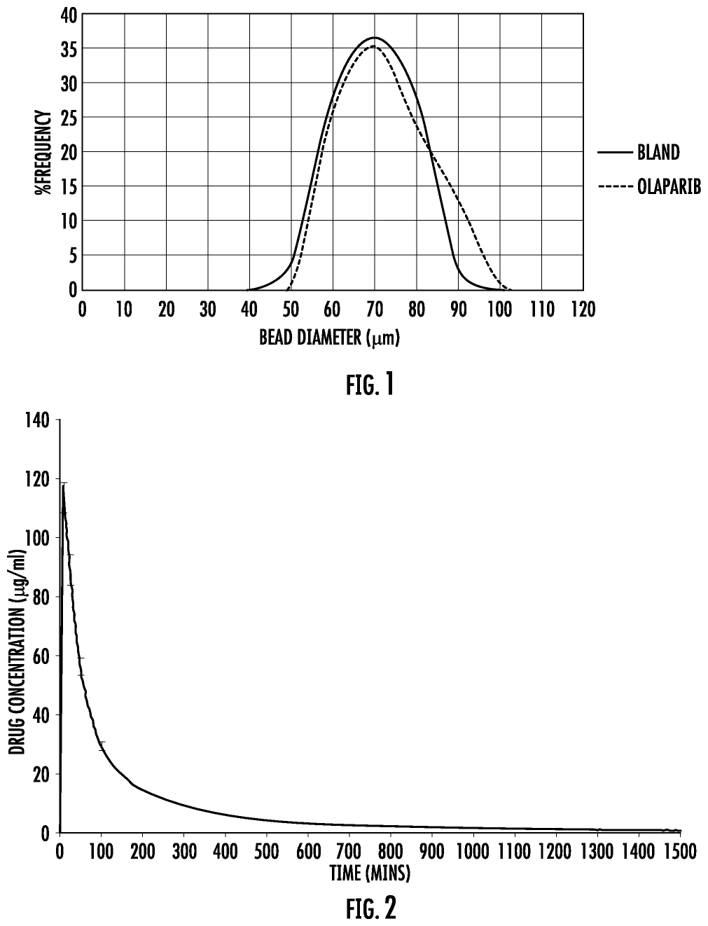

Reconstitution in water resulted in normal appearing smooth spherical microspheres. The size distribution of control non loaded (bland) and loaded microspheres was determined microscopically. The results are illustrated in FIG. 1, which compares sizing histograms of each. After olaparib loading, the microsphere size distribution was not significantly altered.

### Example 4: Elution of Olaparib from Loaded Microspheres

Flow through method: Reconstituted olaparib-loaded microspheres from Example 3, were packed in a flow through system and drug release was determined in a constant flow of warm saline (flow rate 1.7 mL/min at 37° C.) according to Swaine et al., E. J. Pharm. Sci. (2016) 93, 351-359. Olaparib concentration in the column eluate was determined in situ at a wavelength of 279 nm in a flow cell by passage through a UV-visible spectrophotometer.

FIGS. 2 and 3 illustrate the elution profile in terms of olaparib concentration and dose, respectively. Olaparib elution was essentially complete within 5-6 hrs.

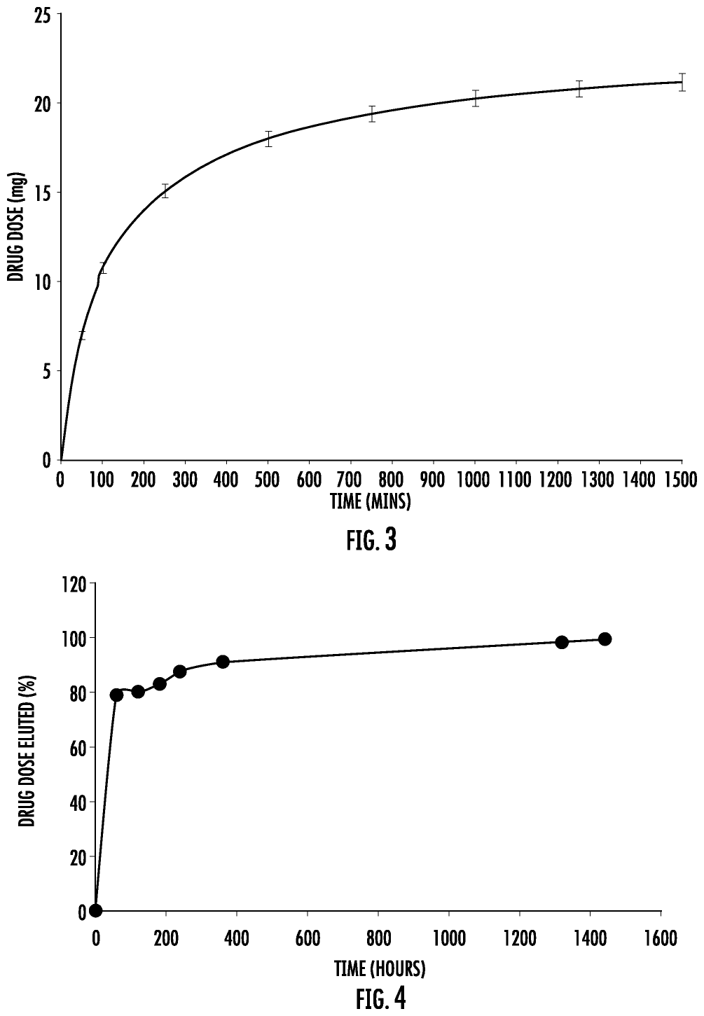

Pseudo sink, jar method (USP2 method): Olaparib loaded microspheres (75-150 uM) were placed in 400 mL of PBS under magnetic stirring at 37° C. After 1 hr, 5 mL of solution was exchanged for fresh PBS and the olaparib concentration measured in the sample by HPLC. Subsequent 1 hr samples were of 200 mL. The data obtained is illustrated in FIG. 4.

### Example 5: Suspension and Catheter Delivery

One vial of Olaparib-loaded microspheres from Example 3 was reconstituted with 1 mL of water followed by 9 mL of Omnipaque® 350. The microspheres were delivered through a 24G (1.7 Fr, 0.7 mm×19 mm) microcatheter successfully without blockage in the microcatheter.

### Example 6. Preparation of PLGA/Olaparib Microspheres (60-150 μm)

0.4 g PLGA (Evonik 50/50 4A, Evonik Industries AG, Essen, Germany) and 40 mg of Olaparib (MedChemExpress, Monmouth Junction, N.J., USA) were dissolved in 1.89 g of dichloromethane (DCM) in a glass vial. A 5 mL solution of 2% solids (w/w) PVA (Aldrich, 146k-186k MW, Sigma-Aldrich, Saint Louis, Mo., USA) in DI water in a glass vial was stirred at 200 rpm using a magnetic stir bar. The PLGA-olaparib solution in DCM was added drop-wise to the stirring PVA solution. The resulting microsphere dispersion was allowed to stir for 5 minutes after which time it was transferred to 150 mL of a stirring solution of DI water. The microspheres were allowed to stir at 300 rpm (harden) for 4 hrs at room temp. The resulting microsphere dispersion was then filtered through a 150 μm mesh filter and the filter was rinsed 3× with DI water. The microsphere dispersion was then poured through a 60 μm mesh filter. The microspheres collected on the filter were then dried overnight in a vacuum oven at room temperature.

### Example 7. Measurement of Olaparib Content of Loaded PLGA Microspheres

1 mg of microspheres were dissolved in 10 mL Acetonitrile (ACN) and the resulting solution was scanned using a UV-Vis spectrophotometer. The adsorption of the olaparib at 276 nm was measured. Concentration of olaparib in the microspheres was determined by comparison to a series of olaparib standards. The olaparib content in the PLGA microspheres was determined to be 6.9% (w/w).

### Example 8. Measurement of Olaparib Release from the PLGA Microspheres

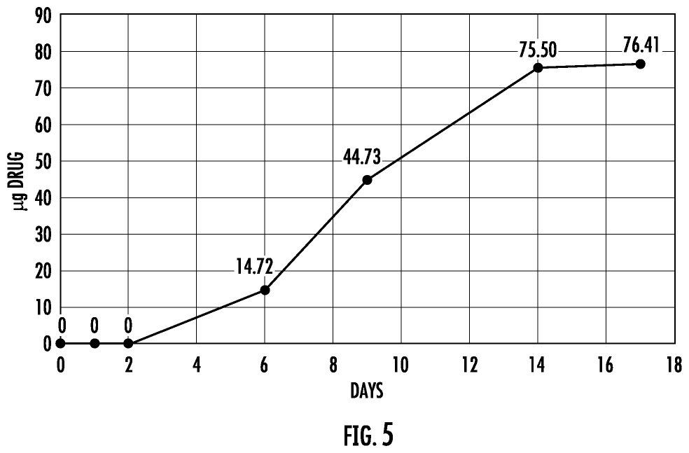

4 mg of microspheres were added to 10 mL pH 7.4 PBS/tween 20 (10 mM phosphate buffer, 140 mM NaCl, 2.7 mM KCl, 0.05% tween 20, Calbiochem, San Diego, Calif., USA) in a 15 mL centrifuge tube. The tube was placed horizontally in an incubator shaker set at 37° C. and 120 prm. The tube was removed at various time points and centrifuged (4000 rpm for 10 sec). The PBS media was removed from the tube and analyzed for olaparib content by UV-Vis at a wavelength 276 nm. 10 mL of fresh PBS was added to the tube and the tube was placed back in the incubator until the next time point. As seen from FIG. 5, olaparib showed sustained release from the PLGA microspheres over a 14-day period.

1. A polymer microsphere comprising a polymer and an inhibitor of the enzyme poly ADP ribose polymerase (PARP inhibitor) wherein the PARP inhibitor is held within the polymer microsphere and is elutable from the microsphere in aqueous media.

2. The polymer microsphere according to claim 1, wherein at least a portion of the PARP inhibitor is held within the microsphere by ionic interaction of the PARP inhibitor with the polymer and/or wherein at least a portion of the PARP inhibitor is physically entrapped within the polymer microsphere.

3. The polymer microsphere according to claim 1, wherein the PARP inhibitor is elutable from the polymer microsphere in phosphate buffered saline (0.01 M phosphate buffered saline (0.138 M NaCl; 0.0027 M KCl), pH 7.4) at 37° C.

4. The polymer microsphere according to claim 1, wherein the polymer has one or a combination of two or more of the following characteristics: (a) the polymer is anionically charged at pH 7.4, (b) the polymer is crosslinked, (c) the polymer is in the form of a hydrogel, or (d) the polymer is biodegradable or bioerodible.

5. The polymer microsphere according to claim 1, wherein the polymer is a polyester, a polysaccharide or a biodegradable PVA polymer.

6. The polymer microsphere according to claim 1, wherein the polymer consists of or comprises poly(lactide-co-glycolide) (PLGA) or is a biodegradable crosslinked PVA.

7. The polymer microsphere according to claim 1, wherein the PARP inhibitor is selected from Olaparib (AZD-2281), Rucaparib (PF-01367338), Niraparib (MK-4827), Talazoparib (BMN-673), Veliparib (ABT-888), CEP 9722, E7016, BGB-290 and 3-aminobenzamide.

8. The polymer microsphere according to claim 1, further comprising a checkpoint inhibitor that is held within the polymer microsphere and is elutable from the microsphere in phosphate buffered saline (0.01 M phosphate buffered saline (0.138 M NaCl; 0.0027 M KCl), pH 7.4) at 37° C.

9. A method for preparing an olaparib loaded hydrogel polymer microsphere comprising the steps of (a) contacting a dehydrated hydrogel polymer microsphere with a solution comprising olaparib dissolved in a first solvent, (b) recovering the microsphere resulting from step (a), and (c) washing the recovered microsphere of step (b) with an second solvent, wherein the first solvent is an organic solvent in which olaparib at a concentration of at least 10 mg/ml at 25° C. and the second solvent is solvent in which olaparib is soluble at a concentration at less than 0.1 mg/ml at 25° C.

10. A method for the treatment of a patient having a solid tumor, comprising delivering to the solid tumor a composition comprising a plurality of polymer microspheres, said microspheres comprising a PARP inhibitor, which is held within the polymer microspheres and is elutable from the polymer microspheres in aqueous media, and wherein the PARP inhibitor is eluted from the polymer microspheres into the tumor tissue at a dose ranging from 1 mg/day to 50 mg/day.

11. The method according to claim 10, wherein the composition is delivered to the tumor by local injection or wherein the composition is delivered to the tumor via one or more blood vessels feeding at least part of the tumor and wherein at least a portion of the polymer microspheres lodge in the blood vessels to provide an embolus.

12. The method of according to claim 10, wherein the polymer microspheres comprise between 0.1 and 50 mg of PARP inhibitor/ml of fully hydrated polymer microspheres

13. The method according to claim 10, wherein the treatment of the tumor additionally comprises treatment with radiation therapy.

14. The method according to claim 13, wherein the radiation therapy comprises external beam radiation therapy (EBRT), brachytherapy or selective internal radiation therapy (SIRT).

15. The method according to claim 10, wherein the treatment of the tumor additionally comprises delivering to the tumor one or more checkpoint inhibitors.

16. The method according to claim 15, wherein the checkpoint inhibitor is selected from inhibitors of the binding of PD-1 to PD-L1, inhibitors of the binding of CTLA-4 to CD80 and/or CD86, inhibitors of the binding of TIGIT to CD-112 and inhibitors of the binding of LAG-3 to MHC class II.

17. The method according to claim 15, wherein the checkpoint inhibitor is selected from antibodies or antigen binding fragments thereof that bind to PD-1, PD-L1, LAG-3, TIM-3, TIGIT or CTLA-4.

18. The method according to claim 15, wherein the checkpoint inhibitor is selected from pembrolizumab, nivolumab, ipilimumab, atezolizumab, avelumab, tremelimumab, relatlimab, durvalumab, etigilimab, domvanalimab, tiragolumab, vibostolimab.

19. The method according to claim 15, wherein the checkpoint inhibitor is selected from antibodies or antigen binding fragments thereof, that bind to TIM-3.

20. The method according to claim 15, wherein the checkpoint inhibitor is selected from LY3321367, MBG453 and TSR-022.
